(19) United States
(12) Patent Application Publication
(10) Pub. No.: US 2005/0054667 A1
(43) Pub. Date: Mar. 10, 2005

(54) Method of treating or inhibiting anti-arrhythmic events in male human patients

(75) Inventors: Werner Cautreels, Weesp (NL); Claus Steinborn, Seelze (DE); Matthias Straub, Barsinghausen (DE); Katrin Beckman, Hannover (DE); Johannes Wilhelmus Catharine Maria Jansen, Culemborg (NL)

Correspondence Address: CROWELL & MORING LLP;INTELLECTUAL PROPERTY GROUP, P.O. BOX 14300, WASHINGTON, DC, 20044-4300, US

(73) Assignee: Solvay Pharmaceuticals GmbH, Hannover (DE)

(21) Appl. No.: 10/894,364

(22) Filed: Jul. 20, 2004

Related U.S. Application Data
(60) Provisional application No. 60/488,346, filed on Jul. 21, 2003.

(51) Int. Cl. A61K 31/4745

(52) U.S. Cl. 514/300

(57) ABSTRACT

A method of treating or inhibiting anti-arrhythmic events in male human patients by administering to a patient in need thereof a pharmaceutically effective amount of a 3,7-diazabicyclo[3,3,1]nonane compound, preferably a 9,9-alkylene-3,7-diazabicyclo[3,3,1]nonane compound, and especially preferably tedisamil, or a physiologically acceptable acid addition salt thereof or a solvate thereof. The method is particularly useful in converting recent onset of atrial fibrillation (Afib) or flutter to normal sinus rhythm (NSR) in male human patients.

### CROSS REFERENCE TO RELATED APPLICATIONS

This application claims priority of U.S. provisional patent application No. 60/488,346, filed Jul. 21, 2003.

### BACKGROUND OF THE INVENTION

The present invention relates to a novel medicinal use of 3,7-diazabicyclo-[3,3,1]nonane compounds, preferably of 9,9-alkylene-3,7-diazabicyclo[3,3,1]-nonane compounds, and most preferably to a novel medicinal use of tedisamil, and of pharmaceutically acceptable acid addition salts and/or solvates of said compounds.

9,9-Alkylene-3,7-diazabicyclononane compounds of formula I and their pharmacological activities are known from published European Patent No. EP 103,833 and the corresponding U.S. Pat. No. 4,550,112, and Finnish Patent No. FI 76,338. Compounds of formula I are a sub-group of the 9,9-N,N′-tetra-substituted 3,7-diazabicyclo[3.3.1]nonane compounds described in the aforementioned patent specifications and can be prepared by the methods described therein. The aforementioned patent specifications disclose that the compounds have useful cardio-active properties, particularly oxygen-saving effects and effects on the heart rate and heart rhythm in general, and are distinguished by a high physiological tolerance. Thus, the compounds show a satisfactory anti-arrhythmic action even at low doses. Moreover, the undesired negative effect on the contractile power of the heart is extremely low; i.e. the compounds have a particularly favorable ratio of anti-arrhythmic or the refractory period of the heart prolonging activities, to negative inotropic secondary activities.

Moreover, Burow et al., U.S. Pat. No. 5,164,401 discloses that the compounds also have a pronounced diuretic effect with a favorable ratio between sodium and potassium excretion.

Furthermore special salts and their manufacture of the 3,7-diazabicyclo[3,3,1]-nonane compounds, in particular of 9,9-alkylene-3,7-diazabicyclo[3,3,1]nonane compounds are described in U.S. Pat. No. 5,324,732. Thus, U.S. Pat. No. 5,324,732 describes fumaric acid salts of said compounds containing 1.5 moles of fumaric acid per mole of the compound. The document provides also a reference to some general pharmacological activities of the compounds of the formula I which are described in the published European Patent No. EP 103,833 and in the corresponding U.S. Pat. No. 4,550,112. The U.S. Pat. No. 4,912,113 also provides new 3,7-diazabicyclo[3,3,1] nonane compounds and their pharmacological properties, and intermediates. However, none of the cited patents addresses any distinction of effects when administering tedisamil to different genders.

It is worthy of mention that the referenced prior art patent documents do not contain any clinical data related to human beings, however all the pharmacological evidence provided is limited to pre-clinical testing in animals like rats and dogs. In addition some of the pre-clinical results are also described in the scientific literature. For example Fischbach et al. describe “Tedisamil in a Chronic Canine Model of Atrial Flutter” (Journal of Cardiovascular Pharmacology, vol. 34, no. 2, August 1999, p. 212 to 218), and also the “Conversion of Atrial Fibrillation by the Experimentasl Antiarrhythmic Drug Tedisamil in Two Canine Models” (Journal of Cardiovascular Electrophsiology, vol. 12, no. 10, October 2001, p. 1138 to 1144). However, Fischbach et al. do not address any distinction of effects when administering tedisamil to different genders. Opie et al. discuss on “Tedisamil in Coronory Disease: Additional Benefits in Therapy of Atrial Fibrillation?” (Journal of Cardiovascular Pharmacology and Therapeutics, vol. 8, no. supplement 1, 2003, p. S 33 to S 37). They give data and figures studies done in the isolated coronary-ligated rat heart, and on the effects of tedisamil on exercise-induced ischemia on diastolic and systolic segment length, and on myocardial oxygen consumption in exercising dogs. It is also stated that tedisamil has anti-ischemic properties, as studied in rat hearts, rabbits, running dogs, and humans; however, Opie et al. do not address any distinction of effects when administering tedisamil to different genders.

Furthermore, Julius Papp et al. have described the “Effects of Bertosamil on Atrial and Ventricular Treshold for Fibrillo-Flutter in Comparison with Quinidine in Anaesthetized Cats” (Pharmaclogical Research, vol. 25, no. supplement 2, 1992, p. 156 to 157), and the “Effects of Bertosamil on Rabbit Atrial and Ventricular Transmembrane Potentials” (Pharmaclogical Research, vol. 25, no. supplement 2, 1992, p. 139 to 140). However, also Papp et al. do not address any distinction of effects when administering tedisamil to different genders.

Although Nicholas A. Flores pertains to clinical phase studies in humans under the title “Tedisamil Solvay” in Current Opinion in Investigational Drugs, vol. 2, no. 1, 2001, p. 97 to 103), no distinction of the effects of tedisamil on different genders is reported.

### SUMMARY OF THE INVENTION

It is the object of the invention to provide a new medical use or a new method of treating male human patients in need of treatment and/or prophylaxis of anti-arrhythmic events.

Another object of the invention is to provide new anti-arrhythmic pharmaceutical compositions having an improved activity profile for the use in male human patients.

The objects of the invention are surprisingly achieved by administering 3,7-diazabicyclo-[3,3,1]nonane compounds, preferably of 9,9-alkylene-3,7-diazabicyclo[3,3,1]-nonane compounds, and most preferably tedisamil, or pharmaceutically acceptable acid addition salts and/or solvates of said compounds to a male human patient in need thereof for the treatment and/or prophylaxis of anti-arrhythmic events such a patient. According to a preferred aspect of the invention, recent onset of atrial fibrillation (Afib) or flutter is converted to normal sinus rhythm (NSR) in male human patients. In accordance with a further aspect of the invention, the objects are achieved by providing an anti-arrhythmic pharmaceutical composition comprising an anti-arrhythmic amount effective in male human patients of at least one anti-arrhythmic active 3,7-diaza-bicyclo[3,3,1]nonane compound as described in the present invention.

The invention therefore relates to the use of the use of 3,7-diaza-bicyclo[3,3,1]nonane compounds, or their physiologically acceptable acid addition salts and/or solvates thereof to produce a pharmaceutical preparation for the treating and/or inhibiting anti-arrhythmic events in male human patients, preferably for converting recent onset of atrial fibrillation (Afib) or flutter to normal sinus rhythm (NSR) in male human patients.

The compounds suitable for this novel medicinal use in male human patients are 3,7-diazabicyclo[3,3,1]nonane compounds corresponding to the Formula I: 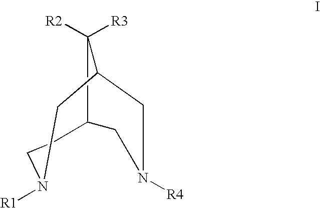
wherein
R1 represents an alkyl group containing from 1 to 6 carbon atoms, an alkylene group containing from 3 to 6 carbon atoms having a double bond which is not linked directly to the nitrogen atom, a cycloalkylalkyl group containing from 4 to 9 carbon atoms, or a benzyl group,
R2 represents a lower alkyl group, and
R3 represents a lower alkyl group, or
R2 and R3 together form an alkylene chain containing from 3 to 6 carbon atoms, and
R4 represents an alkyl group containing from 1 to 6 carbon atoms, an alkenyl group containing from 3 to 6 carbon atoms having a double bond which is not linked directly to the nitrogen atom, a cycloalkylalkyl group containing from 4 to 9 carbon atoms, a group corresponding to the Formula a: 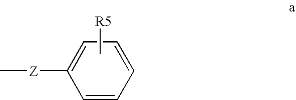
wherein
   R5 represents hydrogen, halogen, lower alkyl or lower alkoxy, and
   Z represents an alkylene chain containing from 1 to 3 carbon atoms or a propenylene chain having a double bond which is conjugated with the phenyl group, or
a group corresponding to the Formula b: 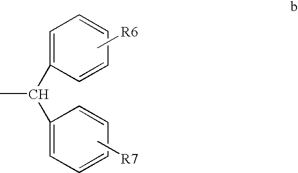
wherein
   R6 represents hydrogen, halogen, lower alkyl or lower alkoxy, and
   R7 represents hydrogen, halogen, lower alkyl or lower alkoxy, or a physiologically acceptable acid addition salt and/or solvate thereof.

Particularly suitable compounds for the novel medicinal use in male human patients according to the invention are compounds of Formula I, in which R1 represents an alkyl group containing from 1 to 6 carbon atoms or a cycloalkylalkyl group containing from 4 to 7 carbon atoms. In further preferred compounds of Formula I, the substituent R4 represents an alkyl group containing from 1 to 6 carbon atoms, a cycloalkylalkyl group containing from 4 to 7 carbon atoms, or a group corresponding to Formula b.

Preferred compounds for the novel medicinal use in male human patients according to the invention are compounds of Formula I, in which R1 represents an alkyl group containing from 3 to 6 carbon atoms or a cycloalkylalkyl group containing from 4 to 7 carbon atoms, and R4 represents an alkyl group containing from 3 to 6 carbon atoms or a cycloalkylalkyl group containing from 4 to 7 carbon atoms. The 3,7-diazabicyclo-[3,3,1]nonane compound may be a 9,9-alkylene-3,7-diazabicyclo[3.3.1]nonane compound of Formula I wherein R2 and R3 together form an alkylene chain containing 4 or 5 carbon atoms, and R1 and R4 independently of one another each denote a straight-chain or branched alkyl group of 3 or 4 carbon atoms or the cyclopropylmethyl group, and physiologically acceptable acid addition salts and/or solvates thereof. Preferred salts for this group of compounds are fumaric acid salts of 9,9-alkylene-3,7-diazabicyclo[3.3.1]nonane compounds containing 1.5 moles of fumaric acid per mole of compound of formula I.

Further preferred compounds for the novel medicinal use in male human patients according to the invention are compounds selected from the group consisting of N,N′-dicyclopropyl-methyl-9,9-tetramethylen-3,7-diazabicyclo[3,3,1]nonane (tedisamil), N-isobutyl-N′-isopropyl-9,9-pentamethylen-3,7-diazabicyclo[3,3,1]nonane, and physiologically acceptable acid addition salts and/or solvates thereof. Preferred salts for this group of compounds are fumaric acid salts of N,N′-dicyclopropylmethyl-9,9-tetramethylene-3,7-diazabicyclo[3,3,1]nonane (tedisamil) or of N-isobutyl-N′-isopropyl-9,9-pentamethylene-3,7-diazabicyclo[3,3,1]nonane containing 1.5 moles of fumaric acid per mole of said 9,9-alkylene-3,7-diazabicyclo[3.3.1]-nonane compound.

Alternatively, as acid addition salts of the 3,7-diazabicyclo[3,3,1]nonane compounds the hydrochloride salts are also very suitable for the novel medicinal use according to the present invention in male human patients.

Particularly preferred 3,7-diazabicyclo[3,3,1]nonane compounds are the 9,9-alkylene-3,7-diazabicyclo[3.3.1]nonane compound tedisamil and the physiologically compatible acid addition salts and/or solvates thereof. These compounds are most preferably used as compounds for the production of pharmaceutical preparations for the treatment and/or inhibition of antiarrhythmic events in male human patients, preferably in conversion of recent onset of atrial fibrillation (Afib) or flutter to normal sinus rhythm (NSR) in male human patients. If a tedisamil acid addition salt is used, it may preferably be used according to the invention in the form of tedisamil hydrochloride or in the form of tedisamil sesquifumarate for the treatment of male human patients. Further pharmacologic-ally compatible acid addition salts of tedisamil are known from European Patent No. EP 103,833. Thus, salts with inorganic acids, e.g. sulfuric acid or hydrohalic acids, especially hydrochloric acid; or with organic acids, for instance lower aliphatic monocarboxylic or dicarboxylic acids such as acetic acid, fumaric acid, tartaric acid, lactic acid, maleic acid, citric acid or salicylic acid; or with sulfonic acids, for instance lower alkyl sulfonic acids such as methane sulfonic acid, or benzene sulfonic acids optionally substituted in the benzene ring by halogen or lower alkyl, such as p-toluene sulfonic acid, are suitable as physiologically acceptable acid addition salts of the compounds of Formula I.

Surprisingly, it has been found that the 3,7,9,9-tetra-substituted 3,7-diazabicyclo[3,3,1]nonane compounds corresponding to Formula I are distinguished by superior effects in anti-arrhythmic male human patients, in particular in the conversion of recent onset of atrial fibrillation (Afib) or flutter to normal sinus rhythm (NSR) in male human patients, in addition to the aforementioned already known general heart-affecting properties. The superior anti-arrhythmic effect of the compounds of Formula I in male human patients, in particular in the conversion of recent onset of atrial fibrillation (Afib) or flutter to normal sinus rhythm (NSR) in male human patients, can be demonstrated by clinical test data with human patients which prove the surprising suitability of 3,7-diazabicyclo[3,3,1]nonane compounds, e.g. of tedisamil and its acid addition salts, for the treatment and/or prophylaxis of anti-arrhythmic effects in male human patients, preferably for conversion of recent onset of atrial fibrillation (Afib) or flutter to normal sinus rhythm (NSR) in male human patients.

### DESCRIPTION OF CLINICAL STUDY DESIGN AND RESULTS

A) Phase II Clinical Study in Humans

The analysis of a first study in humans showed surprisingly that there is a gender difference related to efficacy, e.g. that male human patients show a higher conversion rate compared to women when treated with tedisamil. Furthermore, the male human patients show fewer safety issues than female human patients. Hence, it was surprisingly found that tedisamil as compared to female human patients shows specificity for male human patients in anti-arrhythmic treatment, and in particular in conversion of recent onset of atrial fibrillation (Afib) to normal sinus rhythm (NSR).

This first clinical study in humans was a Multi-Center, Double-Blind, Randomized, Placebo-Controlled, Sequential Ascending Dose Groups Study to evaluate the Efficacy and Safety of Intravenous Tedisamil in the Rapid Conversion to Normal Sinus Rhythm in Patients with Atrial Fibrillation or Flutter. The active ingredient used was tedisamil dihydrochloride. The study was designed as a phase II study and executed in 35 to 40 centers in 3 to 4 countries. Study duration: Screening: up to 48 hours; treatment (in-patient): single 30-minute infusion; safety follow up: 24 hours (in-patient) with continuous telemetry and 28 day safety follow up.

The primary efficacy objective of the study was to demonstrate the superiority of any dose of tedisamil to placebo in the termination of atrial fibrillation/flutter as measured by the percentage of human patients converted to normal sinus rhythm (NSR) (for at least 60 seconds) at any time within 2.5 hours after the start of infusion, in humans. Secondary efficacy objectives were to determine the percentage of human patients remaining in sinus rhythm at 2.5 hours after initiation of the intravenous infusion of tedisamil versus placebo; to determine the percentage of human patients remaining in sinus rhythm at 24 hours after initiation of the intravenous infusion of tedisamil versus placebo; to determine the time to conversion after the start of the infusion of tedisamil versus placebo; and to determine the dose- and plasma concentration-response relationships of tedisamil versus placebo. Safety objective: determining the safety and tolerability of tedisamil versus placebo.

The following methodology for the study in humans was applied: A multi-center, double-blind, randomized, placebo-controlled, sequential ascending dose groups study to evaluate the efficacy and safety of intravenous tedisamil versus placebo. The study drug was infused over 30 minutes, receiving half the dose within 10 minutes and half the dose within the remaining 20 minutes The first patient group received 0.4 mg/kg of body weight, infused as 0.2 mg/kg of body weight within 10 minutes, continued with 0.2 mg/kg of body weight infused within 20 minutes. The next higher dose 0.6 mg/kg of body weight (0.3 mg/kg of body weight infused over 10 min, continued with 0.3 mg/kg of body weight infused over 20 minutes) was only administered after the initial dose has been assessed (blinded) and found to be safe. A third stage may be added with a higher dose. Tedisamil blood concentrations were assessed during the infusion (at 10 and 30 minutes), at conversion to normal sinus rhythm, at recurrence and 24 hours after start of infusion.

Planned Number of Human Subjects: 330 randomized (110 patients per dose group)

Diagnosis and Main Criteria for Inclusion:

The criteria for patient inclusion in the study was atrial fibrillation or flutter with a duration of more than 3 hours and less than 48 hours, occurring as a first or recurrent episode.

Test Product, Dose and Mode of Administration:

Tedisamil (0.4 mg/kg body weight)
Tedisamil (0.6 mg/kg body weight)
   Both dosages were given as an intravenous regimen.
   Reference Therapy, Dose and Mode of Administration:

Placebo (vehicle) administered as a 30-minute intravenous infusion.

Duration of Treatment:

The total infusion time is 30 minutes, with half the dose infused within 10 minutes, and half the dose infused within the remaining 20 minutes.

Criteria for Evaluation:

1) Efficacy:

Primary efficacy: the percentage of human patients converted to normal sinus rhythm (for at least 60 seconds) at any time within 2.5 hours after the initiation of the infusion of study drug.

Secondary efficacy: percentage of human patients in NSR at 2.5 and 24 hours after start of infusion; time to conversion; dose- and concentration-response relationships.

2) Safety:

Physical examination, ECG, 24-hour Holter monitoring, vital signs, laboratory evaluations and adverse events.

Statistical Methods:

All efficacy variables were evaluated separately for human patients with atrial fibrillation and human patients with atrial flutter. Percentages of conversion were compared among treatment groups using a logistic regression model with factors for treatment group and center. Times to conversion were compared among treatment groups using a Cox proportional hazards model with factors for treatment group and center. Dose-response and concentration-response relationships were examined using descriptive statistics.

For the calculation of the number of human patients the following assumptions were made:
1. For atrial fibrillation patients, the percentage of conversion to normal sinus rhythm (at any time within 2.5 hours after the initiation of the infusion) in the placebo group is equal to 20% and the clinically relevant difference is 20%.
2. For atrial flutter patients, the percentage of conversion to normal sinus rhythm in the placebo group is equal to 10% and the clinically relevant difference is 40%.

Interim analyses for efficacy was performed halfway the first and second stages by an external statistician. The purpose is to terminate a stage when the tedisamil dose used in that stage is inefficacious. Technically, each interim analysis was a predictive power calculation for the comparison of tedisamil and placebo with respect to the primary efficacy variable. Blinded safety reviews were performed at the same time as the efficacy analyses and, in addition, at the end of the each stage (if applicable).

B) Phase III Clinical Study in Humans

The analysis of a second study in humans confirmed the finding of the first study, e.g. that there is a gender difference related to efficacy, namely that male human patients show a higher conversion rate compared to women when treated with tedisamil.

This second clinical study in humans was a Multi-Center, Double-Blind, Randomized, Placebo-Controlled, Parallel Design Study to evaluate the Efficacy and Safety of Intravenous Tedisamil Sesquifumarate in the Rapid Conversion to Normal Sinus Rhythm in Subjects with Recent Onset Atrial Fibrillation or Flutter.

The active ingredient used was tedisamil sesqifumarate. The study was designed as a phase II study and executed in 30 to 40 centers in 5 countries. Study duration: Screening: up to 48 hours; treatment (in-patient): single 30-minute infusion; safety follow up: 24 hours (in-patient) with continuous telemetry and 28 day safety follow up.

The primary efficacy objective of the study in humans was to demonstrate the superiority of any dose of tedisamil sesquifumarate to placebo in the rapid conversion to normal sinus rhythm (for at least 60 seconds), as measured by the percentage of subjects converted at any time within 2.5 hours after the start of infusion. Secondary efficacy objectives were to determine the percentage of subjects converting to normal sinus rhythm at any time within 2.5 hours after start of the intravenous infusion and in normal sinus rhythm at 2.5 hours after initiation of the infusion of tedisamil sesquifumarate versus placebo; to determine the percentage of subjects converting to normal sinus rhythm at any time within 2.5 hours after start of the intravenous infusion and in normal sinus rhythm at 24 hours after initiation of the infusion of tedisamil sesquifumarate versus placebo; to determine the percentage of human subjects converting to normal sinus rhythm at any time within 2.5 hours after start of the intravenous infusion and in normal sinus rhythm at hospital discharge; to determine the time to conversion to normal sinus rhythm after the start of the infusion of tedisamil sesquifumarate versus placebo; to determine the dose- and concentration-response relationships of tedisamil sesquifumarate versus placebo; and to determine the energy required for DC cardioversion of tedisamil sesquifumarate versus placebo. Safety objective: determining the safety and tolerability of tedisamil sesquifumarate versus placebo.

The following methodology was applied: A multi-center, double-blind, randomized, placebo-controlled study to evaluate the efficacy and safety of intravenous tedisamil sesquifumarate versus placebo. The study drug was infused over 30 minutes, receiving half the dose within 10 minutes and half the dose within the remaining 20 minutes. Subjects were randomly assigned to receive either:
0.32 mg tedisamil free base per kg bodyweight (bw) (0.16 mg/kg of body weight within 10 minutes, followed by 0.16 mg/kg of body weight within 20 minutes); or
0.48 mg tedisamil free base per kg of body weight (0.24 mg/kg of body weight within 10 min, followed by 0.24 mg/kg of body weight within 20 minutes); or
0.64 mg tedisamil free base per kg of body weight (0.32 mg/kg of body weight within 10 min, followed by 0.32 mg/kg of body weight within 20 minutes); or
a 30 minute placebo infusion.
   Planned Number of Human Subjects:

212 randomized atrial fibrillation subjects (53 human subjects per treatment group). These subjects are the primary target population. In addition, 80 (20 per treatment group) subjects with atrial flutter considered to be enrolled within the scope of this study. However, the study could be terminated without all atrial flutter subjects enrolled if the planned number of atrial fibrillation subjects has been reached.

Diagnosis and Main Criteria for Inclusion:

The patient inclusion criteria were atrial fibrillation or flutter with a duration of greater than 3 hours and less than 45 days, occurring as a first or recurrent episode.

Test Product, Dose and Mode of Administration:

Tedisamil free base 0.32 mg/kg body weight (equivalent to 0.51 mg/kg tedisamil sesquifumarate and to 0.4 mg/kg tedisamil dihydrochloride
Tedisamil free base 0.48 mg/kg body weight (equivalent to 0.77 mg/kg tedisamil sesquifumarate and to 0.6 mg/kg tedisamil dihydrochloride
Tedisamil free base 0.64 mg/kg body weight (equivalent to 1.02 mg/kg tedisamil sesquifumarate and to 0.8 mg/kg tedisamil dihydrochloride
The dosages will be given as an intravenous regimen.
Tedisamil doses in the protocol refer to tedisamil free base.
   Reference Therapy, Dose and Mode of Administration:

Placebo (vehicle) administered as a 30-minute intravenous infusion similar to tedisamil infusion.

Duration of Treatment:

The total infusion time is 30 minutes, with half the dose infused within 10 minutes, and half the dose infused within the remaining 20 minutes.

Criteria for Evaluation:

1) Efficacy:

Primary efficacy: the percentage of human subjects converted to normal sinus rhythm (for at least 60 seconds) at any time within 2.5 hours after the initiation of the infusion of study drug.

Secondary efficacy: percentage of human subjects in normal sinus rhythm at any time within 2.5 hours and at 24 hours after start of infusion, as well as at hospital discharge, time to conversion, dose- and concentration-response relationships and DC cardioversion energy.

2) Safety:

Physical examination, ECG, 24-hour Holter monitoring, vital signs, laboratory evaluations and adverse events.

Statistical Methods:

Percentages of conversion will be compared among treatment groups using the (Pearson) chi-square statistics. Times to conversion will be compared among treatment groups using the log-rank test. Dose-response and concentration-response relationships and the energy required for DC conversion will be examined using descriptive statistics.

Subjects with atrial fibrillation and atrial flutter will be separately analyzed. In addition, both populations will be pooled for analysis. All analyses involving the atrial flutter subject population will be considered as exploratory.

TABLE I
Conversion of recent onset of atrial fibrillation (Afib) to NSR
Conversion to NSR at any time within 2.5 hrs after
start of infusion. ITT human patient sample;
human patients with DC cardioversion are
excluded.

| | | Antiarrhythmic Treatment with | | |
|---|---|---|---|---|
| | Gender | Tedisamil | | |
| | (Humans) | 0.4 mg/kg | 0.6 mg/kg | Placebo |
| Atrial | Male | 18/35 (51.4%) | 18/26 (69.2%) | 2/24 (8.3%) |
| Fibrillation | | | | |
| | Female | 6/17 (35.3%) | 6/16 (37.5%) | 2/22 (9.1%) |
| Atrial Flutter | Male | 1/7 (14.3%) | 1/5 (20.0%) | 0/10 |
| | Female | 0/2 | 2/6 (33.3%) | 0/3 |
| Overall | Male | 19/42 (45.2%) | 19/31 (61.3%) | 2/34 (5.9%) |
| | Female | 6/19 (31.6%) | 8/22 (36.4%) | 2/25 (8.0%) |

TABLE II
Conversion of recent onset of atrial fibrillation (Afib) to NSR
Conversion to NSR at any time within 2.5 hrs after
start of infusion. Human patients with atrial fibrillation
ITT human patient sample; human patients with DC
cardioversion are excluded.

| | | Antiarrhythmic Treatment with | | |
|---|---|---|---|---|
| Gender | | Tedisamil | | |
| (Humans) | Age | 0.4 mg/kg | 0.6 mg/kg | Placebo |
| Male | <65 yrs | 10/20 (50.0%) | 13/18 (72.2%) | 1/14 (7.1%) |
| | >=65 yrs | 8/15 (51.4%) | 5/8 (62.5%) | 1/10 (10%) |
| | Total | 18/35 (51.4%) | 18/26 (69.2%) | 2/24 (8.3%) |
| Female | <65 yrs | 2/5 (40.0%) | 1/3 (33.3%) | 1/5 (20.0%) |
| | >=65 yrs | 4/12 (33.3%) | 5/13 (38.5%) | 1/17 (5.9%) |
| | Total | 6/17 (35.3%) | 6/16 (37.5%) | 2/22 (9.1%) |

TABLE III
Conversion of recent onset of atrial fibrillation (Afib) to NSR
Conversion to NSR at any time within 2.5 hrs after
start of infusion. ITT human patient sample; human
patients with DC cardioversion are
excluded.

| | | Antiarrhythmic Treatment with | | |
|---|---|---|---|---|
| | Gender | Tedisamil | | |
| | (Humans) | 0.4 mg/kg | 0.6 mg/kg | Placebo |
| Atrial | Male | 18/35 (51.4%) | 18/26 (69.2%) | 2/24 (8.3%) |
| Fibrillation | p-value, | <0.001 | <0.001 | |
| | ChiSq (vs | | | |
| | placebo) | | | |
| | Female | 6/17 (35.3%) | 6/16 (37.5%) | 2/22 (9.1%) |
| | p-value, | <0.045 | <0.034 | |
| | ChiSq (vs | | | |
| | placebo) | | | |
| Atrial Flutter | Male | 1/7 (14.3%) | 1/5 (20.0%) | 0/10 |
| | p-value, | 0.218 | 0.143 | |
| | ChiSq (vs | | | |
| | placebo) | | | |
| | Female | 0/2 | 2/6 (33.3%) | 0/3 |
| | p-value, | — | 0.257 | |
| | ChiSq (vs | | | |
| | placebo) | | | |
| Overall | Male | 19/42 (45.2%) | 19/31 (61.3%) | 2/34 (5.9%) |
| | p-value, | <0.001 | <0.001 | |
| | ChiSq (vs | | | |
| | placebo) | | | |
| | Female | 6/19 (31.6%) | 8/22 (36.4%) | 2/25 (8.0%) |
| | p-value, | <0.045 | <0.018 | |
| | ChiSq (vs | | | |
| | placebo) | | | |

It can be seen from these data that the anti-arrhythmic male human patients respond to the treatment with tedisamil more specifically than female human patients in the anti-arrhythmic treatment, and in particular in the conversion of recent onset of atrial fibrillation (Afib)/flutter to normal sinus rhythm (NSR).

What is particularly surprising is the effectiveness of 3,7-diazabicyclo-[3,3,1]nonane compounds, preferably of 9,9-alkylene-3,7-diazabicyclo[3,3,1]-nonane compounds, and most preferably of tedisamil, and of pharmaceutically acceptable acid addition salts and/or solvates in male human patients, as proved by the above results of the studies in humans, in the treatment and/or prophylaxis of anti-arrhythmic events in male human patients, in particular in the conversion of recent onset of atrial fibrillation (Afib) to NSR in male human patients, since such human gender difference related to efficacy has never been observed before in a vast variety of investigations with tedisamil in both human genders, and in particular this gender specificity has neither been observed in pre-clinical animal studies, as described in the state of the art.

From the results regarding efficacy of tedisamil found in the studies with human patients it may be summarized that tedisamil-like 3,7-diazabicyclo-[3,3,1]nonane compounds, preferably of 9,9-alkylene-3,7-diazabicyclo[3,3,1]-nonane compounds, and most preferably tedisamil itself, as well as the acid addition salts, show higher conversion rates in male human patients as compared to female human patients. Extension of infusion beyond the times indicated above did not produce higher conversion rates. From the results it may be summarized regarding safety: Almost all observed TdP (Torsade de Point) in 30 min regimen were in female human patients. Only one male human patient showed TdP with high dosage on 0.72 in 3114 study.

As a therapeutic agent, 3,7-diazabicyclo-[3,3,1]nonane compounds, preferably of 9,9-alkylene-3,7-diazabicyclo[3,3,1]-nonane compounds, and most preferably of tedisamil, and of pharmaceutically acceptable acid addition salts and/or solvates, may be contained according to the invention, together with conventional pharmaceutical auxiliaries and/or carriers, in solid or liquid pharmaceutical preparations dedicated to the administration in humans. Examples of solid preparations are preparations which can be administered orally, such as tablets, coated tablets, capsules, powders or granules, or alternatively suppositories. These preparations may contain conventional pharmaceutical inorganic and/or organic carriers, such as talcum, lactose or starch, in addition to conventional pharmaceutical auxiliaries, for example lubricants or tablet disintegrating agents. Liquid preparations such as suspensions or emulsions of 3,7-diazabicyclo-[3,3,1]nonane compounds, preferably of 9,9-alkylene-3,7-diazabicyclo[3,3,1]-nonane compounds, and most preferably of tedisamil, and of pharmaceutically acceptable acid addition salts and/or solvates thereof, may contain the usual diluents such as water, oils and/or suspension agents such as polyethylene glycols and the like. Other auxiliaries may additionally be added, such as preservatives, taste correctives and the like.

The 3,7-diazabicyclo-[3,3,1]nonane compounds, preferably of 9,9-alkylene-3,7-diazabicyclo[3,3,1]-nonane compounds, and most preferably tedisamil, and pharmaceutically acceptable acid addition salts and/or solvates thereof, can be mixed and formulated with the pharmaceutical auxiliaries and/or carriers in known manner. For the production of solid medicament forms dedicated to human beings, 3,7-diazabicyclo-[3,3,1]nonane compounds, preferably of 9,9-alkylene-3,7-diazabicyclo[3,3,1]-nonane compounds, and most preferably tedisamil, and pharmaceutically acceptable acid addition salts and/or solvates thereof, can for example be mixed with the auxiliaries and/or carriers in conventional manner and can be wet or dry granulated. The granules or powder can be poured directly into capsules or be pressed into tablet cores in conventional manner. These can be coated in known manner if desired.

### EXAMPLES

The following Examples 1 to 3 describe pharmaceutical preparations according to the invention which contain an active substance of Formula I, and also the production of such pharmaceutical preparations. The following examples explain the production of pharmaceutical preparations containing tedisamil dihydrochloride. Pharmaceutical preparations containing tedisamil sesquifumarate may be obtained in an analogous manner.

### Example 1

### Tablet Composition

| | | |
|---|---|---|
| | 20 parts | of N,N′-dicyclopropylmethyl-9,9-tetramethylen-3,7- |
| | | diazabicyclo[3,3,1]-nonane dihydrochloride |
| | 30 parts | of corn starch |
| | 55 parts | of lactose |
| | 5 parts | of polyvinylpyrrolidone |
| | 2 parts | of magnesium stearate |
| | 3 parts | of talcum |
| Total | 115 parts | |

### PREPARATION METHOD

The active substance was mixed with corn starch and finely powdered lactose in a mixer. The resulting mixture was thoroughly moistened with a 20% solution of polyvinylpyrrolidone (“Kollidon 25”, from BASF) in deionized water. If necessary, additional deionized water was added. The moist granules were passed through a 2 mm sieve, dried on trays at 40 DEG C. and then passed through a 1 mm sieve (Frewitt machine). After the granules had been mixed with magnesium stearate and talcum, tablets weighing 115 mg were pressed therefrom, so that each tablet contained 20 mg of the active substance.

### Example 2

### Capsules Composition

| | | |
|---|---|---|
| | 20 parts | of N-isobutyl-N′-isopropyl-9,9-pentamethylen-3,7- |
| | | diazabicyclo[3,3,1]nonane dihydrogen fumarate |
| | 20 parts | of corn starch |
| | 45 parts | of lactose |
| | 3 parts | of polyvinylpyrrolidone |
| | 1.5 parts | of magnesium stearate |
| | 0.5 parts | of highly dispersed silicic acid |
| Total | 90 parts | |

### PREPARATION METHOD

The active substance was mixed with corn starch and finely powdered lactose in a mixer. The resulting mixture was thoroughly moistened with a 20% solution of polyvinylpyrrolidone (“Kollidon 25”, from BASF) in deionized water. If necessary, deionized water was added. The moist granules were passed through a 1.6 mm sieve (Frewitt machine), dried on trays at 40 DEG C., and then passed through a 1 mm sieve (Frewitt). After the granules had been mixed with magnesium stearate and highly dispersed silicic acid (“Aerosil 200”, from Degussa), 90 mg thereof in each case were filled by means of an automatic encapsulating machine into size 4 hard gelatin capsules, so that each capsule contained 20 mg of active substance.

### Example 3

### Ampoules Composition (Per Ampoule)

| | |
|---|---|
| 5 mg | N,N′-dicyclopropylmethyl-9,9-tetramethylen-3,7- |
| | diazabicyclo[3,3,1]nonane dihydrochloride |
| 16 mg | Sodium chloride |

Water for injection purposes to make up to 2.0 ml

### PREPARATION METHOD

Sodium chloride was dissolved in water for injection purposes. The active substance was added and dissolved while stirring. Sufficient water for injection purposes was added to make up the final volume. The mixture was passed through a 0.25 .mu. membrane filter. 2.15 ml aliquots were filled into brown glass ampoules, and the ampoules were hermetically closed. The ampoules were sterilized with steam for 30 minutes at 121 DEG C. 2 ml of the resulting injection solution contains 5 mg of the active substance.

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

1. A method of treating or inhibiting anti-arrhythmic events in a male human patient, said method comprising administering to a patient in need thereof a pharmaceutically effective amount of a 3,7-diazabicyclo[3,3,1]nonane compound or a physiologically acceptable acid addition salt thereof or a physiologically acceptable solvate thereof.

2. A method according to claim 1, wherein the anti-arrhythmic event is recent onset of atrial fibrillation (Afib) or flutter, and the atrial fibrillation or flutter is converted to normal sinus rhythm (NSR).

3. A method according to claim 1, wherein the the 3,7-diazabicyclo[3,3,1]nonane compound corresponds to Formula I: 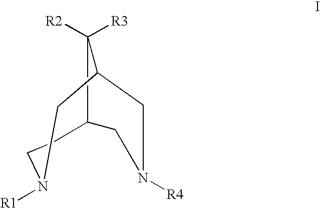
wherein
R1 represents an alkyl group containing from 1 to 6 carbon atoms, an alkylene group containing from 3 to 6 carbon atoms having a double bond which is not linked directly to the nitrogen atom, a cycloalkylalkyl group containing from 4 to 9 carbon atoms, or a benzyl group,
R2 represents a lower alkyl group, and
R3 represents a lower alkyl group, or
R2 and R3 together form an alkylene chain containing from 3 to 6 carbon atoms, and
R4 represents an alkyl group containing from 1 to 6 carbon atoms, an alkenyl group containing from 3 to 6 carbon atoms having a double bond which is not linked directly to the nitrogen atom, a cycloalkylalkyl group containing from 4 to 9 carbon atoms,
a group corresponding to the Formula a: 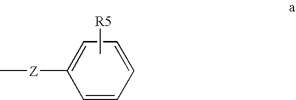
wherein
R5 represents hydrogen, halogen, lower alkyl or lower alkoxy, and
Z represents an alkylene chain containing from 1 to 3 carbon atoms or a propenylene chain having a double bond which is conjugated with the phenyl group, or
a group corresponding to the Formula b: 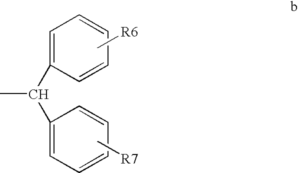
wherein
R6 represents hydrogen, halogen, lower alkyl or lower alkoxy, and
R7 represents hydrogen, halogen, lower alkyl or lower alkoxy.

4. A method according to claim 1, wherein R1 represents an alkyl group containing from 1 to 6 carbon atoms or a cycloalkylalkyl group containing from 4 to 7 carbon atoms.

5. A method according to claim 1, wherein R4 represents an alkyl group containing from 1 to 6 carbon atoms, a cycloalkylalkyl group containing from 4 to 7 carbon atoms, or a group corresponding to Formula b.

6. A method according to claim 1, wherein R1 represents an alkyl group containing from 3 to 6 carbon atoms or a cycloalkylalkyl group containing from 4 to 7 carbon atoms, and R4 represents an alkyl group containing from 3 to 6 carbon atoms or a cycloalkylalkyl group containing from 4 to 7 carbon atoms.

7. A method according to claim 1, wherein the 3,7-diazabicyclo[3,3,1]nonane compound is a 9,9-alkylene-3,7-diazabicyclo[3.3.1]nonane compound, wherein R2 and R3 together form an alkylene chain containing 4 or 5 carbon atoms, and R1 and R4 each independently denote a straight-chain or branched alkyl group of 3 or 4 carbon atoms or the cyclopropylmethyl group.

8. A method according to claim 7, wherein the 9,9-alkylene-3,7-diazabicyclo[3.3.1]-nonane compound is a fumaric acid salt containing 1.5 moles of fumaric acid per mole of compound of formula I.

9. A method according to claim 1, wherein the 3,7-diazabicyclo[3,3,1]nonane compound is selected from the group consisting of N,N′-dicyclopropylmethyl-9,9-tetramethylen-3,7-diazabicyclo[3,3,1]nonane, N-isobutyl-N′-isopropyl-9,9-pentamethylen-3,7-diazabicyclo[3,3,1]nonane, physiologically acceptable acid addition salts thereof, and physiologically acceptable solvates thereof.

10. A method according to claim 9, wherein said 3,7-diazabicyclo[3,3,1]nonane compound is a fumaric acid salt of N,N′-dicyclopropylmethyl-9,9-tetramethylene-3,7-diazabicyclo[3,3,1]nonane or of N-isobutyl-N′-isopropyl-9,9-pentamethylene-3,7-diazabicyclo[3,3,1]nonane containing 1.5 moles of fumaric acid per mole of compound of formula I.

11. A method according to claim 1, wherein the 3,7-diazabicyclo[3,3,1]nonane compound is a hydrochloride salt.

12. A method according to claim 6, wherein the 3,7-diazabicyclo[3,3,1]nonane compound is a hydrochloride salt.

13. A method according to claim 8, wherein the 3,7-diazabicyclo[3,3,1]nonane compound is a hydrochloride salt.
